(19) United States
(12) Patent Application Publication
(10) Pub. No.: US 2017/0333599 A1
(43) Pub. Date: Nov. 23, 2017

(54) OSTEOINDUCTIVE NANOFIBER SCAFFOLD FOR BONE REGENERATION

(71) Applicant: RTI Surgical, Inc., Alachua, FL (US)

(72) Inventors: Amanda B. LoGuidice, Gainesville, FL (US); Alison P. Houlihan, Gainesville, FL (US)

(21) Appl. No.: 15/599,152

(22) Filed: May 18, 2017

Related U.S. Application Data
(60) Provisional application No. 62/338,425, filed on May 18, 2016.

(51) Int. Cl. A61L 27/46; A61L 27/44; A61L 27/58; D01D 5/00; A61L 27/18; A61L 27/38; A61L 27/54

(52) U.S. Cl. CPC A61L 27/46; A61L 27/3834; A61L 27/3847; A61L 27/446; A61L 27/54; D01D 5/0007; A61L 27/18; A61L 27/58; A61L 2300/112; A61L 2300/412; A61L 2400/12; A61L 2430/02; A61L 2300/106

(57) ABSTRACT

The present application is directed to the field of scaffolds for tissue engineering. The scaffolds are typically comprised of nanofibers and are optionally biomineralized. The present application provides a process for forming nanofibrous materials via electrospinning and for biomineralizing such materials. The scaffolds of the present application can be biomineralized and contain a plurality of cells either on or within the scaffold, resulting in synthetic, bioresorbable scaffolds that can be used in various biomedical applications, such as for bone regeneration.

### RELATED APPLICATIONS

[Not Applicable]

### FEDERALLY SPONSORED RESEARCH OR DEVELOPMENT

[Not Applicable]

### FIELD OF TECHNOLOGY

The present application is directed to the field of synthetic, bioresorbable scaffolds for tissue engineering. The scaffolds are typically comprised of nanofibers and are optionally biomineralized. The present application provides a process for forming nanofibrous materials and for biomineralizing such materials.

### BRIEF SUMMARY

In one aspect, the application is directed to a synthetic, bioresorbable, osteoinductive scaffold comprising, in combination, nanofibers composed of polycaprolactone (PCL) and polyethylene glycol diacrylate (PEGDA). In a further aspect, the nanofiber scaffold is biomineralized and comprises a plurality of cells on or within the scaffold.

The present application is further directed to nanofiber scaffolds containing a plurality of cells comprising progenitor cells, stem cells, connective tissue cells, chondrocytes or osteoblasts wherein said plurality of cells; scaffolds comprise stem cells in certain embodiments.

The present application is further directed to nanofiber scaffolds comprising a nanofiber mat or sheet. The nanofibers of the scaffolds can be randomly oriented, of uniform diameter and without significant fiber deformation.

The present application is further directed to scaffolds comprising nanofibers having a concentration of PCL in the nanofibers of about 40% to 95% by weight. In other embodiments, the concentration of PEGDA in the nanofibers is about 5% to 60% by weight. In further embodiments the concentration of PCL/PEGDA in the nanofibers is about 50/50 (by weight) or about 75/25 (by weight).

The present application is further directed to nanofiber scaffolds where the nanofibers are created by electrospinning. In further embodiments, the solution concentration of PCL used for electrospinning is 2-30% (wt/v) in dichloromethane. In other embodiments, the solution concentration of PCL used for electrospinning is 5-15% (wt/v) in dichloromethane.

The present application is further directed to nanofiber scaffolds that are biomineralized using a multi-step process. In certain embodiments, the multi-step process uses serial immersions in CaCl₂ and Na₂HPO₄; and in certain embodiments the serial immersions includes at least 3 cycles.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIGS. 1A-1C show nanofibers with increasing PCL concentration. FIG. 1A shows nanofibers made from electrospun solutions of 7.5% PCL and 25% PEGDA. FIG. 1b shows nanofibers made from electrospun solutions of 9.75% PCL and 25% PEGDA. FIG. 1C shows nanofibers made from electrospun solutions of 11.25% PCL and 25% PEGDA. Scale bar is 200 μm.

FIG. 2 shows light microscopy for nanofiber uniformity and orientation of nanofibers made from solutions of 9.75% PCL and 25% PEGDA. Scale bar is 50 μm.

FIGS. 3A-3C show alizarin red staining of scaffolds subjected to various treatments. FIG. 3A shows no biomineralization. FIG. 3B shows biomineralization via one step (batch) method. FIG. 3C shows biomineralization via multi step serial biomineralization. Scale bar is 500 μm.

FIG. 4 shows cell viability on a nanofiber scaffold. Dead cells are shown as red dots and live cells are shown as green dots. In this study, viability was ˜94% after 7 days.

FIGS. 5A and 5B show MAPC expression of ALP. FIG. 5A shows expression at 24 hours. FIG. 5B shows expression at 7 days. MAPC in osteogeneic media (OM), MAPC in AMD, MAPC+nanofiber scaffold (NFS) in AMD and MAPC+nanofiber scaffold (NFS) in OM are compared. AMD is a cell media proprietary to Athersys, Inc. When compared to all other groups, n=2, p<0.05, unless otherwise indicated. The asterisks indicate to which test groups the p-values for significance specifically relates.

FIGS. 6A and 6B show MAPC expression of osteonectin. FIG. 6A shows expression at 10 days. FIG. 6B shows expression at 28 days. When compared to MAPC in osteogenic media (OM) and MAPC+nanofiber scaffold (NFS) in AMD, n=2, p<0.05. AMD is a cell media proprietary to Athersys, Inc. The asterisks indicate to which test groups the p-values for significance specifically relates.

### DETAILED DESCRIPTION

In the field of bone tissue engineering, recent focus has been on composites combining polymeric matrices with inorganic components. These composite grafts are often seeded with growth factors and signaling molecules to augment osteogenic differentiation. However, elevated and/or prolonged dosage of these inductive molecules can have undesirable cytotoxic and inflammatory effects.

Nanofibrous scaffolds have potential for use in tissue engineering applications due to their structural similarity to natural extracellular matrix (ECM), which consists of nanofibers ranging in diameter from 50 to 500 nm. It is believed that cells are better able to attach to and organize around fibers with smaller diameters, and studies have shown that cells respond acutely to physical stimuli in the nanoscale range. In addition, electrospun nanofibers have high surface area-to-volume ratios, allowing for maximal cellular attachment and proliferation.

In one aspect the application is directed to synthetic, bioresorbable scaffolds for tissue engineering that promotes cell (e.g. osteogenic) differentiation in vivo, without the use of supplemental proteins and growth factors. In vivo efficacy of such scaffolds may be enhanced through the incorporation of cells on or in the scaffold.

This application provides a novel scaffold comprised of nanofibers that is optionally biomineralized. The present application provides a process for forming nanofibrous materials via electrospinning and for biomineralizing such materials. In certain embodiments scaffolds contain cells on or within the scaffold. The scaffolds are typically comprised of electrospun nanofibers and are optionally biomineralized. Nanofibrous scaffolds can be biomineralized to enhance their bioactivity. Biomineralization is a process to mineralize a material using certain mineralization factors, and, in this instance, is a way to mimic the structure of bone.

Scaffolds for tissue engineering can take many forms. The current application uses electrospun fibers to create a nominally 2-D or 3-D network. Such scaffolds function as a substrate for cell growth and aid in the healing and development of new tissue. Scaffolds that are biomineralized can be osteoinductive and aid in bone formation and/or regeneration. Scaffolds can additionally be layered for other tissue engineering applications. Cell-seeded scaffolds provide delivery of cells to the body for a myriad of purposes depending on the cell type. Also contemplated are scaffolds that function as a wrap and/or covering to enclose grafts or implants.

As used herein the term “bioresorbable” is intended to refer to materials that can dissolve, be broken down by or be absorbed by the body. Bioresorbable materials usually do not have to be removed after implantation, which is advantageous to the patient.

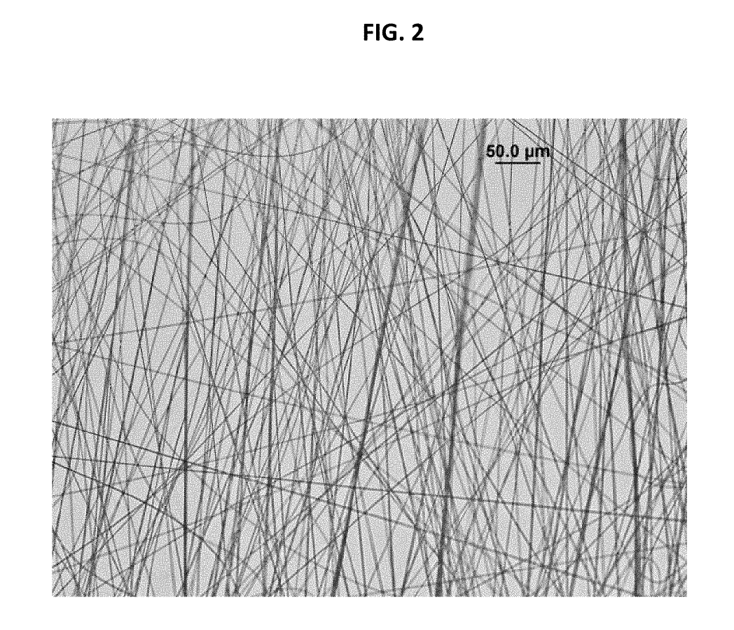

In another aspect, the application is directed to a process for the formation of nanofibers. The nanofibers are composed of two or more polymer components that create a bioresorbable scaffold. The scaffold is created by electrospinning. The process of electrospinning creates scaffolds with interwoven or woven fibers. That is, the fibers have random orientation, as depicted, for example, in FIG. 2.

The nanofibers of the application are created by electrospinning. As compared to conventional fiber spinning, which produces fibers in the micrometer range, electrospinning produces fibers in the nanometer diameter range. Fibers with diameters around 500 nm or below are considered nanoscale. Such fibers are called nanofibers. The fibers comprising the scaffold of the application are within the range of 10-500 nm. In brief, electrospinning uses an electric field generated between a polymer solution (usually in a syringe) and a target to create nanoscale fibers of a polymer that are collected on the target surface. The fiber alignment and diameter can be varied by changing the electrospinning parameters.

Polymer solutions are prepared for electrospinning and loaded into appropriate delivery vehicle, e.g. a syringe. Solution concentrations of polymers control the final fiber composition, but are affected by solvent evaporation during processing.

Nanofibers created by electrospinning can be collected in various ways, including, but not limited to, stationary plate, rotating mandrel and collection into an ice bath. This allows for greater volume of scaffold to be collected. Nanofibers are collected as mats or sheets; properties vary by adjusting the spinning parameters, such as collector distance, applied voltage, pump speed, and speed of rotation. The nanofibers can be further modified to enhance their properties. For example, additional post-processing modifications can be made, such as chemical crosslinking and surface coating.

The molecular weights of the polymers used in the electrospinning process affect the properties of the fibers. Additionally, the solvent and viscosity of the solution that is used for electrospinning, along with flow rate through the syringe can also affect fiber composition and morphology. Changing the electric field during electrospinning can change the properties of the nanofibers generated. Properties such as length, thickness and fiber orientation can be modulated.

One way to modify nanofibers is via biomineralization. Biomineralization is mediated in nature by extracellular proteins. Biomimetic biomineralization occurs by using chemicals to mimic the natural process. To create materials that aid in bone regeneration, calcium containing compounds are commonly used. Biomineralized biomaterials can promote osteogenesis, osteoinductivity and/or osteoconductivity.

The nanofibers of the invention, whether biomineralized or not, can also contain a cellular component. The cellular component comprises a plurality of cells on or within the scaffold. When used with cells capable of promoting bone growth, these scaffolds can promote osteoblast differentiation and thus are osteoinductive. An osteoinductive substance has at least some ability to promote or assist in bone growth, such as the ability to recruit and transform cells from the host which have the potential for repairing bone tissue. For example, demineralized bone matrix and osteoinductive proteins such as bone morphogenetic proteins (BMPs) are considered to be osteoinductive substances. Autograft, allograft, xenograft or recombinantly produced BMPs or other naturally produced or recombinant growth factors are also considered osteoinductive substances. Osteoinductive proteins include some of the proteins in the transforming growth factor-beta (TGF-beta) superfamily of proteins, which includes the bone morphogenetic proteins (BMPs), activins and inhibins.

In certain embodiments, the scaffold comprises a plurality of cells on or within the scaffold. Plurality of cells includes but is not limited to progenitor cells, stem cells, connective tissue cells, chondrocytes or osteoblasts. In certain embodiments, adult stem cells are utilized. Other cell types can be utilized when the scaffolds of this application are used in non-bone applications. For example, in an embodiment directed to wound care, adipose stem cells could be used.

In one embodiment, progenitor cells are used as the cellular component. In certain embodiments, multipotent adult progenitor class cells (MAPC® or MAPC-Class Cells®), a specific type of naturally occurring adult stem cell with recognized properties, are utilized. The MAPC technology is proprietary to Athersys, Inc. and represents a distinctive type of stem cell with recognized angiogenic and immuno-modulatory properties. MAPC-class cells have the ability to form any of the three germ layers: mesoderm, ectoderm and endoderm. Since the cells are multipotent in nature, they have the capability to differentiate along the chondrogenic, adipogenic or osteogenic lineage (mesoderm layer). Similar to mesenchymal stem cells, MAPC are stem cells found in bone marrow. Both have the potential to differentiate into multiple specialized cell types. Both also have recognized osteogenic properties and are non-immunogenic (meaning they do not elicit an immune rejection response).

However, MAPC-class cells provide higher levels of select angiogenic proteins important to promote revascularization or new blood vessel formation, a necessary component of bone healing. Additionally, MAPC-class cells impact endothelial cells' affinity to modulate white blood cell migration more effectively than mesenchymal stem cells. This can reduce the presence of immune cells at the injury site and result in an attenuated inflammatory response.

MAPC-class cells are nonimmunogenic. They do not elicit an immune rejection response. MAPC-class cells do not express HLA class II antigens. MAPC-class cells also attenuate the proliferation of T-cells in an in vitro model. MAPC-class cells have shown potential for reducing local inflammation by regulating the production of inflammatory cytokines. Expression of the pro-inflammatory cytokine TNF(alpha) was downregulated in the presence of MAPC-class cells conditioned media.

MAPC-class cells show alkaline phosphatase activity and mineralization when in osteogenic media. Alkaline phosphatase is an enzyme produced by osteoblasts and is used as an early marker of osteogenesis. Mineralization is the last stage of new bone formation and is used as a late marker of osteogenesis.

MAPC-class cells secrete higher levels of select angiogenic proteins than MSCs. IL-8 (Interleukin 8) and CXCL-5 (epithelial derived neutrophil activating peptide 78) values are significantly higher for MAPC-class cells. Angiogenesis supports revascularization and is vital for achieving successful bone regeneration and fracture healing. Another angiogenic property is tube formation. MAPC-class cells demonstrated denser, more well-defined tube formations when compared to mesenchymal stem cells in vitro. MAPC-class cells influenced endothelial cells (the cells responsible for blood vessel formation) to form closed wall tubes—resembling the inner diameter of a blood vessel wall responsible for blood vessel formation. Blood Vessel Formation in vivo is also shown to be significantly higher for MAPC-class cells.

In an animal model (rat), viable MAPC-class cells were present at the site of implant for at least 10 days post-op. MAPC-class cells were only present at sites where they were implanted.

The present scaffolds can be osteoconductive as well as osteoinductive. When a substance is osteoconductive, it has at least some ability to provide support for the growth of new host bone. For example, demineralized bone matrix, intact bone allografts, calcium phosphate and hydroxyapatite are considered to be osteoconductive substances. The present scaffolds can also be osteogenic as well as osteoinductive. When a substance is osteogenic, it includes cells such as osteoblasts that can form bone, or stem cells that can be turned into bone-forming cells.

Nanofiber scaffolds of the present application provide favorable environments for cell viability, promoting cellular growth. On both biomineralized and non-biomineralized scaffolds, cell attachment occurs within hours, and cell viability remains high over time. In some embodiments using biomineralized scaffolds, cell attachment occurred within about 1-10 hours, and viability was above 80% after 7 days. In other embodiments, cell attachment occurred within about 2-5 hours, and viability was above 90% after 7 days. In other embodiments, cell attachment occurred within about 2-3 hours, and viability was above 95% after 7 days. In other embodiments, cell attachment occurs within about 2 hours, and viability was about 99% after 7 days.

There are a variety of physical configurations of the scaffolds that are contemplated, such as woven nanofiber mats and sheets. Additionally, these scaffolds can be used alone or in conjunction with other materials. The terms “graft” and “implant” can be used to refer to materials for implantation in a human. The terms “graft” and “implant” are used interchangeably herein.

“Implant” (or “graft”), as used herein, refers to any material the implantation of which into a human or an animal is considered to be beneficial. Accordingly, the implant may be tissue-derived material, such as bone, skin, and the like, or it may be a metallic or synthetic material having an external surface or internal structure that may require cleaning, sterilization or passivation. An implant may comprise autograft tissue, allograft tissue, xenograft tissue or combinations thereof, and in the case of mineralized tissues, such as bone, the implant may comprise mineralized tissue, partially demineralized tissue, completely demineralized tissue, and combinations thereof. The implant may comprise unitary or monolithic graft material, assembled bone materials such as those described in U.S. patent application Ser. Nos. 09/782,594 and 09/941,154, shaped implants such as those described in U.S. Pat. Nos. 6,440,444 and 6,696,073, and allogeneic biocompatible matrices such as those described in U.S. patent application Ser. Nos. 10/754,310 and 10/793,976. The present processes and apparatus may also be employed in the treatment of implants such as those described in U.S. Pat. Nos. D461,248; 6,290,718; 6,497,726; 6,652,592; 6,685,626; and 6,699,252. All of the foregoing patents and patent applications are incorporated by reference herein.

Suitable polymers for use in the present electrospinning processes include, but are not limited to crosslinkable polymers including polycaprolactone (PCL), polyethylene glycol diacrylate (PEGDA), poly(lactic-co-glycolic acid) (PLGA) and poly(methyl methacrylate) (PMMA) and blends and copolymers thereof. Polymers may be crosslinkable or non-crosslinkable, with crosslinkable polymers utilized in certain embodiments. Polymers for electrospinning must be in solution. Various organic solvents such as dichloromethane (methylene chloride), tetrahydrofuran, dimethylformamide, choloroform, ethanol, isopropanol (and other alcohols) can be utilized to solubilize the polymers for electrospinning. Water and various aqueous acids and bases can also be used in certain applications. In certain embodiments, dichloromethane is utilized.

In certain embodiments, polycaprolactone (PCL) is utilized (specifically poly(ε-caprolactone)), and the concentration of PCL in the nanofibers is about 40% to 95% by weight, where a second polymer makes up the remainder. In other embodiments, polyethylene glycol diacrylate (PEGDA) is utilized, and the concentration of PEGDA in the nanofibers is about 5% to 60% by weight. In certain embodiments, the nanofibers comprise both polycaprolactone (PCL) and polyethylene glycol diacrylate (PEGDA). In certain embodiments, concentration ratios are 50% PCL/50% PEGDA or 75% PCL/25% PEGDA (by weight). These nanofibers are created via electrospinning solutions of PCL and PEGDA in dichloromethane, or another appropriate solvent.

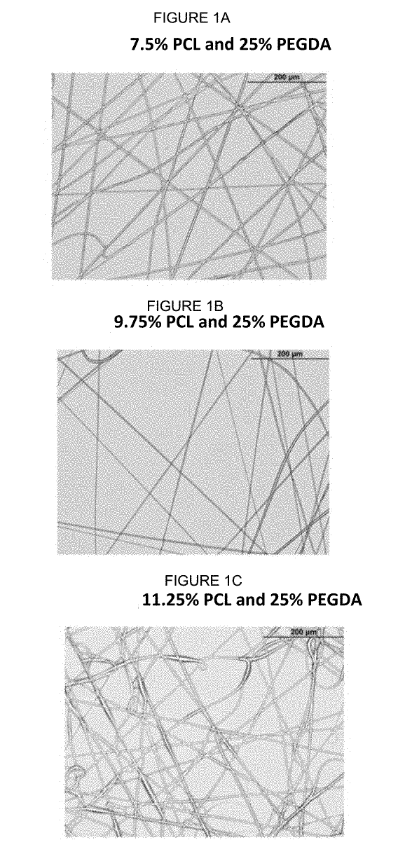

Initial concentrations in solution of PCL that are used for the electrospinning process can be varied between about 2-30% (wt/v) in dichloromethane. In one embodiment, solution concentration of PCL in dichloromethane is between about 5-15%. In certain embodiments, concentrations are about 7.5%, about 9.75% and about 11.25%. See FIGS. 1A-1C where scaffolds created using various solution concentrations of PCL are shown.

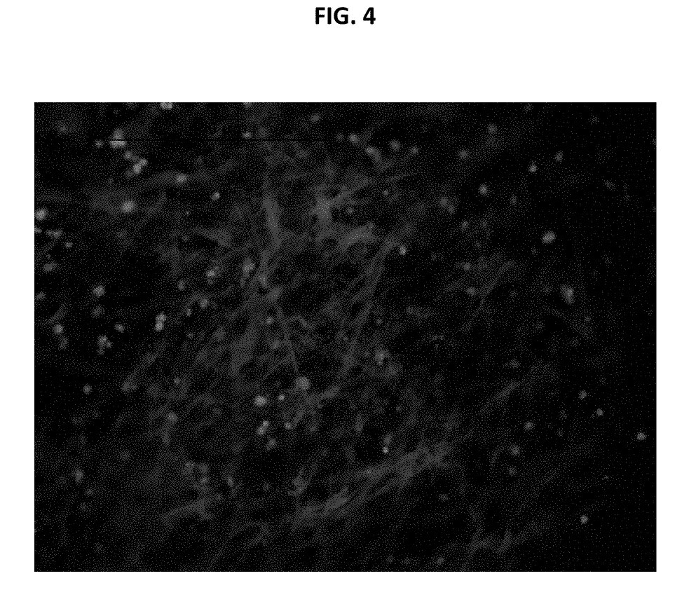

Initial concentrations in solution of PEGDA that are used for the electrospinning process can be varied between about 5-40% (wt/v) in dichloromethane. In one embodiment, concentration of PEGDA in dichloromethane is between about 20-30%. In certain embodiments, concentrations are about 25%. The nanofiber scaffolds made from electrospinning a 9.75% PCL/25% PEGDA solution produced randomly oriented nanofibers of uniform diameter and without significant fiber deformation. See FIG. 2. Cell viability on the scaffolds made with 9.75% PCL was about 94% after 7 days in culture; see Example 3 and FIG. 4.

After nanofibers are synthesized, a ceramic such as hydroxyapatite or β-tricalcium phosphate can be incorporated via biomineralization. In certain embodiments, biomineralization processes may include a serial immersion in biomineralization agents or a one-step (batch) process. Both procedures are described in Example 2. A calcium containing agent is used for biomineralization. In certain embodiments, calcium chloride is utilized but other agents are contemplated.

Possible biomineralization agents for use include but are not limited to calcium chloride (CaCl₂), calcium carbonate (CaCO₃) and hydroxyapatite, also called HA (Ca₅(PO₄)₃(OH), or alternatively Ca₁₀(PO₄)₆(OH)₂)). The biomineralization process can result in particles of mineralized material that exist on or in the scaffold. Such mineral particles (e.g. hydroxyapatite) can be nanoparticlulate/nanocrystalline in size. Usually for bone tissue engineering, mineral particles produced by biomineralization comprise hydroxyapatite and/or calcium phosphate.

The biomineralization agent may be provided in a solution or mixture, usually using DI water as a solvent. For biomineralization via a multi-step process of series immersion, about 50-500 mM CaCl₂ and 50-300 mM Na₂HPO₄ are used (aqueous solutions). Other ranges such as 100-500 mM CaCl₂ and 100-300 mM Na2HPO4 or 100-400 mM CaCl₂ and 100-200 mM Na₂HPO₄ can be utilized. In one embodiment, 200 mM CaCl₂ and 120 mM Na₂HPO₄ is utilized. Incubation times, number of repeats, as well as specific concentrations of the chemicals can be modified for specific performance. In certain embodiments, incubation times are about 1 hour in the calcium containing solution (CaCl₂), followed by 1 hour in Na₂HPO₄. Scaffolds can be rinsed between solutions, with water or other suitable solvents. These cycles can be repeated, in some embodiments, 2-5 times, until desired biomineralization levels are reached. Desirable biomineralization was achieved by this method using a total of three cycles.

For biomineralization via a one-step (batch) process, a simulated body fluid was utilized to biomineralized scaffolds with calcium phosphate. Such fluids are known in the art and can comprise sodium chloride (NaCl), potassium chloride (KCl) and magnesium chloride (MgCl₂) (see, Tas, A. C. and S. B. Bhaduri, Rapid coating of Ti6Al4V at room temperature with a calcium phosphate solution similar to 10× simulated body fluid. Journal of Materials Research, 2004. 19(09): p. 2742-2749.) In certain embodiments, concentrations comprise about 500-1500 mM NaCl, 1-10 mM KCl, 1-50 mM CaCl₂.2H₂O, 1-10 mM MgCl₂.6H₂O, 1-50 mM Na₂HPO₄. In one embodiment, 116.8860 g NaCl, 0.7456 g KCl, 7.3508 g CaCl₂.2H₂O, 2.0330 g MgCl₂.6H₂O, and 2.3996 g Na₂HPO₄ are used. Expressed as molarity, concentrations of reagents in this embodiment are 1000 mM NaCl, 5 mM KCl, 25 mM CaCl₂.2H₂O, 5 mM MgCl₂.6H2O, 10 mM Na₂HPO₄.

Biomineralized scaffolds can be evaluated for their mineral content. Testing (alizarin red staining) shows that the amount mineral deposition on scaffolds is higher when they are treated using the multi-step serial biomineralization method as compared to the one-step (batch) processes. Thus, in some embodiments, biomineralization via the multi-step process (series immersion) described above is specifically contemplated.

Biomineralized nanofibrous scaffolds present a tunable, low-cost approach to synthetic osteoinductive bone matrix fabrication. The scaffold has potential application for a variety of uses in bone tissue engineering, resulting from its versatility and its ability to promote cell (e.g. osteogenic) differentiation without embedded growth factors and signaling molecules. The absence of growth factors mitigates many of the risks associated with the delivery of signaling proteins. Thus, in some embodiments, a scaffold without embedded growth factors or signaling molecules is contemplated.

After biomineralization the nanofibers are dried. The nanofibers may be air dried, dried in an oven or lyophilized. At this point the nanofibers are in the form of a nanofiber mat or nanofiber sheet. Maximum thickness is on the order of millimeters or less; in one embodiment the nanofiber sheets/mats are about 1 mm or less; in other embodiments the nanofiber sheets/mats are about 0.5 mm or less. The nanofiber sheets/mats can be thicker or thinner depending on the duration of electrospinning. In some embodiments thicknesses of less than 2 mm or less than about 2.0 mm is desired, alternatively 1.5 mm or about 1.5 mm, alternatively 1.0 mm or about 1.0 mm, alternatively 0.5 mm or about 0.5 mm, alternatively less than 0.5 mm or less than about 0.5 mm. Electrospinning parameters can be increased to create thicker sheets and/or nanofiber sheets can be layered to create implants of greater thickness.

The scaffolds of the present application, when presented as an implant for use in a human, can have various shapes including that of a strip, a sheet, a film, a disk, a molded 3D shaped object, a plug, a sponge, and a gasket. Any of these objects may include a cavity, a pouch, a hole, a post, a hook, or a suture.

For a strip in this embodiment, the dimensions are typically from about 10 mm to 500 mm long, by 10 mm to 200 mm wide. In another embodiment, from about 15 mm to 200 mm long by 15 mm to 100 mm wide; in a further embodiment from about 50 mm to 90 mm long by 15 mm to 35 mm wide. The cross section of such a strip may take any shape including a rectangle, square, triangle, other polygon, circle, half circle, ellipse, or partial ellipse.

For a sheet in this embodiment, the dimensions are typically from about 20 mm to 300 mm by 10 mm to 100 mm. In another embodiment, from about 30 mm to 150 mm by 20 mm to 70 mm. In a further embodiment, from about 50 mm to 100 mm by 25 mm to 50 mm.

For a film in this embodiment, the dimensions are typically from about 20 mm to 300 mm by 10 mm to 100 mm. In another embodiment, from about 30 mm to 150 mm by 20 mm to 70 mm. In a further embodiment, from about 50 mm to 100 mm by 25 mm to 50 mm. A film can be created by removing most air bubbles prior to or while molding a thin layer of material. The film shape may be flat or may follow a 3D contoured shape. The film may be created in a single layer or by laminating multiple layers of material.

For a disk in this embodiment, the dimensions are typically from about 10 mm to 100 mm diameter; in other embodiments, from about 30 mm to 80 mm diameter or about 55 mm to 65 mm diameter. A disk can be described as a cylinder with nominal diameter greater than nominal height.

For a molded 3D shaped object in this embodiment, the nominal outer body dimensions are typically up to about 100 mm by 100 mm. In other embodiments, up to about 50 mm by 70 mm or up to about 30 mm by 50 mm. A molded 3D shape is typically defined so as to fit into a particular anatomical feature or surgically created space, such as a bone defect, dental cavity, a drilled tunnel (or channel) in a bone, or the space between/around/next to vertebral bodies in the spine.

For a plug in this embodiment, the dimensions are typically from about 1 mm to 100 mm diameter and at least 1 mm tall (height). In other embodiments, from about 2 mm to 20 mm diameter or from about 4 mm to 15 mm diameter. Plugs can be up to 40 mm tall (height). A plug may be described as a cylinder or extruded 2D shape, such as a square, triangle, star, or polygon, with nominal height greater than nominal diameter or characteristic width of the 2D shape.

For a sponge in this embodiment, the dimensions are typically up to about 500 mm by 500 mm. In other embodiments, up to about 100 mm by 100 mm. In further embodiments, up to about 50 mm by 50 mm.

For a gasket in this embodiment, the outside body dimensions are typically up to about 100 mm by 100 mm. In other embodiments, up to about 50 mm by 50 mm. In further embodiments, up to about 25 mm by 25 mm or 10 mm by 10 mm. The cross section of such a gasket may take any shape including a rectangle, square, triangle, other polygon, circle, half circle, ellipse, or partial ellipse, tracing the entire periphery or some portion of the outside body shape.

Other embodiments include, but are not limited to wraps, bags or patches. In some embodiments, dimensions of the wraps, bags or patches can include 10 mm to 500 mm long, by 10 mm to 200 mm wide; from about 15 mm to 200 mm long by 15 mm to 100 mm wide; from about 50 mm to 90 mm long by 15 mm to 35 mm wide; from about 20 mm to 300 mm by 10 mm to 100 mm; from about 30 mm to 150 mm by 20 mm to 70 mm. In other embodiments, scaffold dimensions of 10 mm², 20 mm², 30 mm², 40 mm², 50 mm², 60 mm², 70 mm^{2,} 80 mm^{2,} 90 mm² or 100 mm² are contemplated. The nanofiber scaffolds can function as a cell delivery system. In certain embodiments, a 3D cell delivery system is contemplated which maintains cell viability and delivers targeted cells to a patient.

Dimensions of electrospun fiber mats or sheets depend on the electrospinning set-up and predetermined parameters. In one embodiment, mandrel dimensions are up to about 150×150 mm, larger or smaller mandrels are also contemplated. In another embodiment, stationary plate dimensions are up to about 300 mm, larger or smaller plates are also contemplated. The electrospinning set-up can be adjusted to achieve larger or smaller scaffolds.

Cells are seeded on nanofiber mats by introducing the cells to the nanofibers in small volumes (about 10-1000 μL, in certain embodiments about 20-50 μI) of medium and allowing them to incubate and attach. Initial conditions are about 37 degrees; low oxygen conditions (about 3%) can be used to assist the process. Amounts of cells to be loaded depend on the application. In some embodiments, at least about 10,000 cells are loaded. In other embodiments, at least about 50,000 cells are loaded. In further embodiments at least about 100,000 cells are loaded. After initial attachment, additional media can be added to the scaffolds to completely soak and provide nutrients to the cells. Via this procedure, cells attached to the nanofiber scaffolds. In one embodiment, cells are attached on the surface of the scaffold. In other embodiments, cells are present on multiple planes indicating penetration through the nanofiber scaffold (mat or sheet).

The nanofibers of the current application can be used as a base scaffold for multiple applications. The nanofibers can be used for other tissue engineering applications such as for wound care or as a surgical mesh. In these embodiments, it is the fibers would not be biomineralized and cells of a different type could be used. Nanofibers of the present application could be used in conjunction with other cell types in non-bone applications, such as surgical meshes. For example, nanofiber scaffolds could be used as epidermal grafts or partial- and full-thickness dermal grafts, for example, for burn care. Burn or epidermal grafts could utilize endothelial cells. The nanofibers can also be used to create layered scaffolds.

Nanofiber scaffolds can have aligned or random fiber orientation. Nanoscale and microscale surface topography of implants can be used to influence the behavior and physiological functions of cells. Contact guidance is a phenomenon by which cells rearrange their cytoskeletons in alignment with the patterns on the surface with which they are in contact. Contact guidance can direct cell elongation and cell migration, as well as physiological processes and behaviors, including differentiation. Altering the alignment of nanofibers within the nanofiber mats exerts influence over the behavior of attached cells, and can direct cell migration and spreading, as well as impact stem cell differentiation efficiency.

Layered scaffolds comprise layers of nanofiber sheets. The layered sheets can comprise nanofibers having different orientations (random/aligned). Also, scaffolds having layered sheets of nanofibers with stem cells in-between the layers is contemplated. Layering sheets of nanofibers serves the purpose of combining different orientations in the same scaffold, and/or of obtaining scaffolds with increased thickness. Scaffolds can be fused or laminated together to make multi-layer constructs. Additionally, scaffolds could be layered with other materials, such as hydrogels. Hydrogels include but are not limited to those such as collagen and poly(ethylene glycol) (PEG).

### EXAMPLES

### Example 1

In one embodiment, a nanofiber scaffold is made by the following method. Polycaprolactone (PCL) was dissolved in dichloromethane (9.75% w/v) then mixed with poly(ethylene glycol) diacrylate (PEGDA) (75:25 v/v). The solution was loaded into a 5 ml syringe and electrospun. The pump speed was 6 ml/h, the applied voltage was 20 kV, and the distance from the syringe to the collecting plate was 17 cm. This process was conducted under a fume hood. 4.5 ml of solution was electrospun, and the resulting woven nanofiber mat was allowed to air dry on the foil overnight. Light microscopy was used to evaluate fiber diameter and morphology. Nanofiber uniformity and orientation of fibers made from a 9.75% PCL-25% PEG is shown in FIG. 2.

### Example 2

### Biomineralization

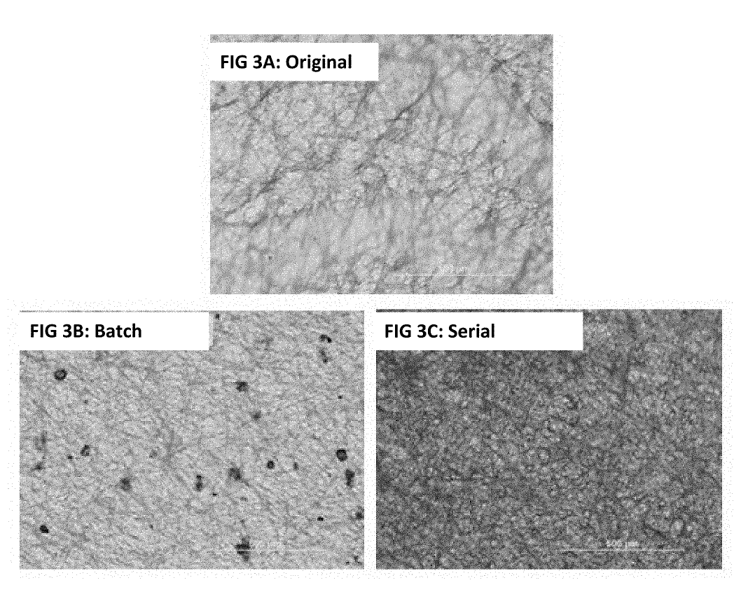

Biomineralization was performed using two methodologies, described in detail below. Alizarin red staining was used to qualitatively evaluate mineral deposition on the scaffold during the biomineralization process. Alizarin red staining indicated higher mineral deposition on scaffolds treated using the serial biomineralization method (FIG. 3C) when compared with scaffolds biomineralized using the one-step (batch) process (FIG. 3B) and untreated scaffolds (FIG. 3A).

Biomineralization via Multi Step Series Immersion

Scaffolds such as those prepared in Example 1 were incubated for 1 hour in 200 mM CaCl₂ (MW 110.98 g/mol), followed by 1 hour in 120 mM Na₂HPO₄ (MW 141.96 g/mol). Scaffolds were briefly rinsed in ddH₂O between solutions. This was repeated twice for a total of three cycles (6 hours).

Biomineralization in a One-Step (Batch) Process

Scaffolds such as those prepared in Example 1 were incubated for 6 hours in simulated body fluid (SBF) according to methods outlined by Tas and Bhaduri (Tas, A. C. and S. B. Bhaduri, Rapid coating of Ti₆Al₄V at room temperature with a calcium phosphate solution similar to 10× simulated body fluid. Journal of Materials Research, 2004. 19(09): p. 2742-2749). Briefly, 1.9 L of ddH₂O was added to a 2 ml glass beaker on a stir plate. Reagents were added in the following order: 116.8860 g NaCl, 0.7456 g KCl, 7.3508 g CaCl2.2H₂O, 2.0330 g MgCl₂.6H₂O, and 2.3996 g Na₂HPO₄. Each reagent was fully dissolved before addition of the next. ddH20 was added to bring the solution volume to 2 L. The pH was adjusted to 4.35-4.40. Prior to use, 200 ml of this stock solution was added to a glass beaker on a stir plate. NaHCO₃ was added until pH reached ˜6.5. Scaffolds were fully immersed for 6 hours at 37° C.

### Example 3

### Cell Viability Study

In order to ensure that the scaffolds can contain viable cells, a cell viability study was undertaken. Poly(ε-caprolactone)/poly(ethylene glycol) diacrylate nanofibers were fabricated by electrospinning as indicated in Example 1. The nanofiber scaffolds (NFS) were biomineralized in CaCl₂ and Na₂HPO₄ as indicated in Example 2 (multi-step method). The nanofiber mats are air dried, soaked in ethanol (70%) and then dried again. Samples were then soaked in cell culture medium before cell seeding.

Multipotent adult progenitor cell (MAPC® or MAPC-Class Cells®) attachment and viability were evaluated using calcein and ethidium bromide staining. Biomineralized scaffolds were seeded with cells for 4 weeks to evaluate in vitro osteogenic differentiation. Early differentiation was assessed via alkaline phosphatase (ALP) expression through day 7. Osteonectin expression was measured at 10 and 28 days via enzyme-linked immunosorbent assay (ELISA).

MAPC were loaded in 10 μI onto each of 4 nanofiber scaffolds (such as those from Example 2) cut to 20 mm². The cell seeding procedure specifically comprised pipetting the cells at a total of 100,000 cells in small volume (10 μL) of medium. Cells were incubated for 2 hours (at 37 degrees, low Oxygen conditions (3%)), then 2 ml of basal culture medium was added to each well. Cells were cultured under hypoxic conditions for 7 days. After 7 days, the cells were washed in DPBS with 1% anti-anti, and then stained with the Life Technologies LIVE/DEAD® Viability/Cytotoxicity Kit for mammalian cells for 30 minutes at room temperature. Cells were imaged using a Leica fluorescent microscope.

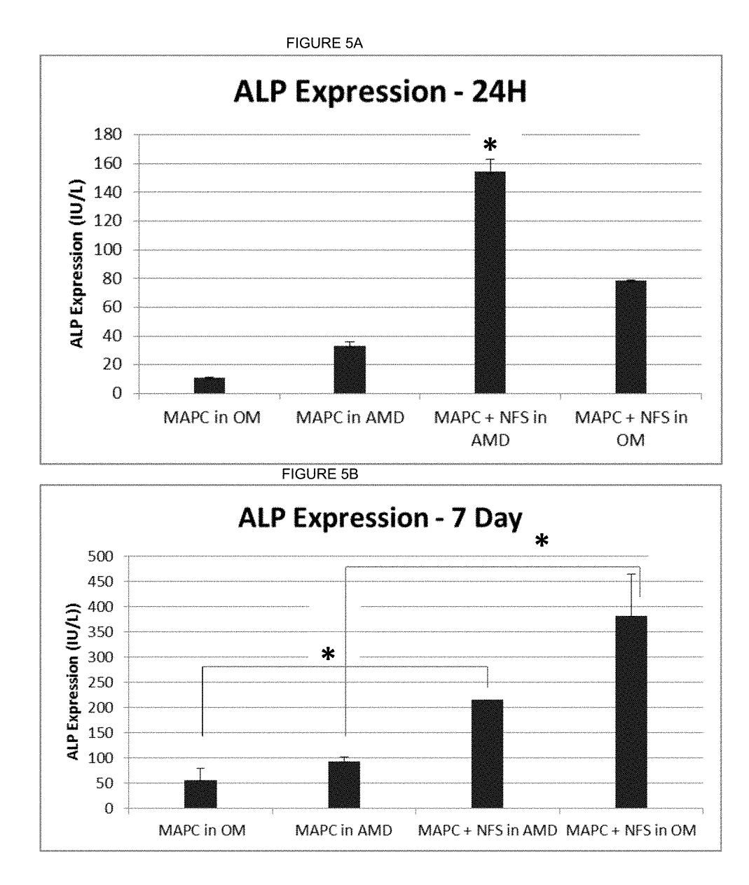

Cell viability is demonstrated in FIG. 4. Dead cells are shown as red dots and live cells are shown as green dots. In this study, viability was ˜94% after 7 days. In vitro differentiation studies also demonstrated an increase in the early bone marker, ALP, during the first 7 days of cell culture on biomineralized scaffolds, with and without inductive medium. See FIG. 5.

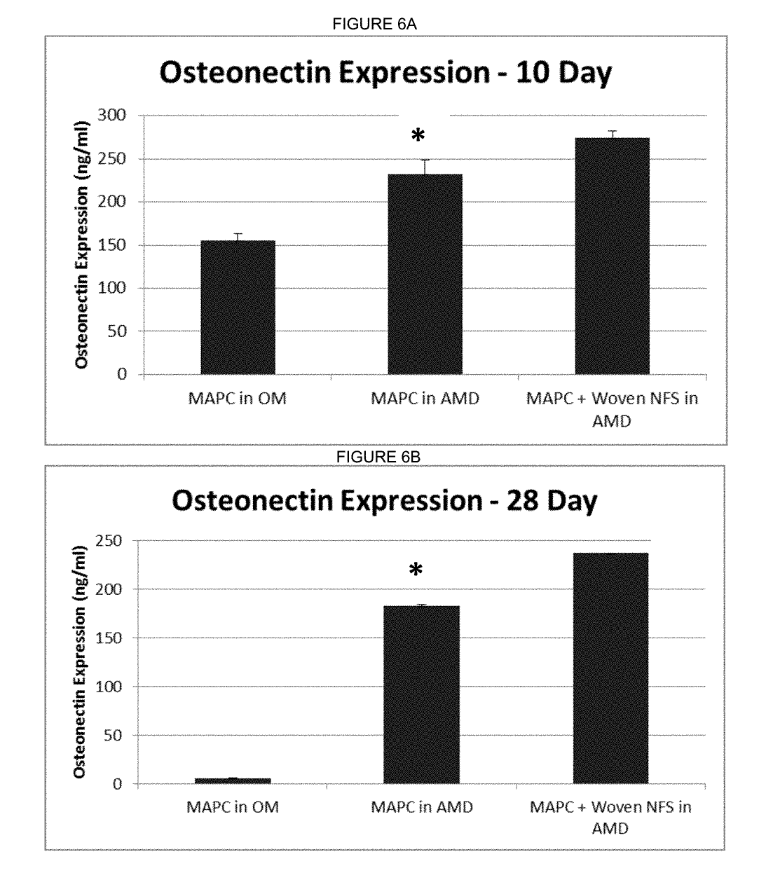

Osteonectin expression levels, analyzed via ELISA, indicated enhanced osteogenic differentiation on the biomineralized scaffolds at 10 and 28 days, when compared to control groups. See FIG. 6. Biomineralized scaffolds increased stem cell expression of osteonectin without the use of inductive medium.

All patents, test procedures, and other documents cited herein, including priority documents, are fully incorporated by reference to the extent such disclosure is not inconsistent with this application and for all jurisdictions in which such incorporation is permitted.

### BIBLIOGRAPHY

Kim, H. W., H. H. Lee, and J. C. Knowles, Electrospinning biomedical nanocomposite fibers of hydroxyapatite/poly(lactic acid) for bone regeneration. J Biomed Mater Res A, 2006. 79(3): p. 643-9.

Fujihara, K., M. Kotaki, and S. Ramakrishna, Guided bone regeneration membrane made of polycaprolactone/calcium carbonate composite nano-fibers. Biomaterials, 2005. 26(19): p. 4139-47.

Chatterjea, A., et al., Clinical application of human mesenchymal stromal cells for bone tissue engineering. Stem Cells Int, 2010. 2010: p. 215625.

Arosarena, O. A., et al., Comparison of bone morphogenetic protein-2 and osteoactivin for mesenchymal cell differentiation: effects of bolus and continuous administration. J Cell Physiol, 2011. 226(11): p. 2943-52.

Hunziker, E. B., et al., Osseointegration: the slow delivery of BMP-2 enhances osteoinductivity. Bone, 2012. 51(1): p. 98-106.

Rui, J., et al., Controlled release of vascular endothelial growth factor using poly-lactic-co-glycolic acid microspheres: in vitro characterization and application in polycaprolactone fumarate nerve conduits. Acta Biomater, 2012. 8(2): p. 511-8.

Barnes, C. P., et al., Nanofiber technology: designing the next generation of tissue engineering scaffolds. Adv Drug Deliv Rev, 2007. 59(14): p. 1413-33.

Stevens, M. M. and J. H. George, Exploring and engineering the cell surface interface. Science, 2005. 310(5751): p. 1135-8.

Laurencin, C. T., et al., Tissue engineering: orthopedic applications. Annu Rev Biomed Eng, 1999. 1: p. 19-46.

Eap, S., et al., A living thick nanofibrous implant bifunctionalized with active growth factor and stem cells for bone regeneration. Int J Nanomedicine, 2015. 10: p. 1061-75.

Shanmugavel, S., et al., Precipitation of hydroxyapatite on electrospun polycaprolactone/aloe vera/silk fibroin nanofibrous scaffolds for bone tissue engineering. J Biomater Appl, 2013. 29(1): p. 46-58.

Tas, A. C. and S. B. Bhaduri, Rapid coating of Ti6Al4V at room temperature with a calcium phosphate solution similar to 10× simulated body fluid. Journal of Materials Research, 2004. 19(09): p. 2742-2749.

Ziabari, M. et al. Application of direct tracking method for measuring electrospun nanofiber diameter. Braz J Chem Eng, 2016. 26(1): p. 533-62.

1. A synthetic, bioresorbable, osteoinductive scaffold comprising, in combination, nanofibers composed of polycaprolactone (PCL) and polyethylene glycol diacrylate (PEGDA) and further wherein said scaffold is biomineralized and comprises a plurality of cells on or within the scaffold.

2. The scaffold of claim 1, wherein said plurality of cells comprises progenitor cells, stem cells, connective tissue cells, chondrocytes or osteoblasts.

3. The scaffold of claim 2, wherein said plurality of cells comprises stem cells.

4. The scaffold of claim 1, wherein the scaffold comprises a nanofiber mat or sheet.

5. The scaffold of claim 4, wherein said nanofibers are randomly oriented, of uniform diameter and without significant fiber deformation.

6. The scaffold of claim 1, wherein the concentration of PCL in the nanofibers is about 40% to 95% by weight.

7. The scaffold of claim 4, wherein the concentration of PEGDA in the nanofibers is about 60% to 5% by weight.

8. The scaffold of claim 5, wherein the concentration of PCL/PEGDA in the nanofibers is about 50/50 (by weight).

9. The scaffold of claim 5, wherein the concentration of PCL/PEGDA in the nanofibers is about 75/25 (by weight).

10. The scaffold of claim 1, wherein the nanofibers are created by electrospinning.

11. The scaffold of claim 10, wherein the solution concentration of PCL used for electrospinning is 2-30% (wt/v) in dichloromethane.

12. The scaffold of claim 11, wherein the solution concentration of PCL used for electrospinning is 5-15% (wt/v) in dichloromethane.

13. The scaffold of claim 1, wherein said scaffold is biomineralized using a multi-step process.

14. The scaffold of claim 13, wherein said multi-step process uses serial immersions in CaCl₂ and Na₂HPO₄.

15. The scaffold of claim 14, wherein said serial immersions includes at least 3 cycles.
